(19) United States
(12) Patent Application Publication
(10) Pub. No.: US 2025/0224399 A1
(43) Pub. Date: Jul. 10, 2025

(54) METHOD FOR DETECTING HOMOCYSTEINE IN PLASMA AND METHOD FOR DIAGNOSING GLIOBLASTOMA BY USING NOVEL FLUORESCENT PROBE

(71) Applicant: UNIVERSITY-INDUSTRY COOPERATION GROUP OF KYUNG HEE UNIVERSITY, Yongin-si, Gyeonggi-do (KR)

(72) Inventors: Dokyoung KIM, Seoul (KR); Jongmin AN, Seoul (KR); Jaehoon KIM, Seoul (KR); Youngwoong KIM, Chungju-si (KR)

(73) Assignee: UNIVERSITY-INDUSTRY COOPERATION GROUP OF KYUNG HEE UNIVERSITY, Yongin-si, Gyeonggi-do (KR)

(21) Appl. No.: 18/851,764

(22) PCT Filed: Mar. 21, 2023
(86) PCT No.: PCT/KR2023/003721

§ 371 Date: Sep. 27, 2024

(30) Foreign Application Priority Data
Mar. 29, 2022 (KR) 10-2022-0038670

(51) Int. Cl. G01N 33/574; C07D 413/12; G01N 33/68

(52) U.S. Cl. CPC G01N 33/57407; C07D 413/12; G01N 33/57488; G01N 33/6815

(57) ABSTRACT

The present disclosure relates to a fluorescent probe compound for sensing homocysteine, and use thereof, wherein the fluorescent probe compound prepared according to the present disclosure enables homocysteine present in in vitro plasma and feces to be detected within a short period of time without interference from other biomolecules, and thus has very high usability with respect to bio-derived materials and can be effectively used in early diagnosis identification of glioblastoma.

### TECHNICAL FIELD

The present disclosure relates to a fluorescent probe compound based on pyridine-nitrobenzofurazan, a method for detecting homocysteine in plasma using the same, and a method for early diagnosis of glioblastoma through the same.

### BACKGROUND ART

Glioblastoma is a tumor that arises from neuroglial cells present throughout the brain tissue, and is a fatal brain tumor in humans, accounting for approximately 12 to 15% of brain tumors. The exact cause of glioblastoma has not been found, but it is known to be related to genetic and environmental factors. In addition, glioblastoma is one of the primary malignant brain tumors that occurs in adults. If left untreated after diagnosis, death occurs within 3 to 6 months. Even with treatment, the average survival period is 12 to 14 months, making it a highly malignant tumor. To diagnose glioblastoma, a neurological checkup is performed, followed by examination and diagnosis using imaging diagnostic medical devices such as magnetic resonance imaging (MRI) and computed tomography (CT). However, this checkup and diagnostic method has higher examination costs than other diagnostic methods and the possibility of side effects due to the use of contrast agents. In addition, in cases where glioblastoma is diagnosed through imaging diagnostic medical devices, the glioblastoma is already at an advanced stage, and the timing of glioblastoma treatment is missed or a lot of expense is required for diagnosis and treatment. It is an essential task to overcome these diagnostic shortcomings of glioblastoma in a more convenient way and to diagnose and prevent glioblastoma early.

To date, technologies for detecting glioblastoma have continued to develop imaging diagnostic medical devices (MRI, CT, etc.) and contrast agents. However, contrast agents used for imaging diagnostic medical devices may have side effects, so there are diagnostic shortcomings. In addition, imaging diagnosis is not a simple procedure and is costly. Accordingly, the development of a system for the early diagnosis of glioblastoma that may simplify diagnosis and reduce cost consumption through a noninvasive method is an essential challenge to overcome.

Recent studies have reported the potential of homocysteine as a biomarker for glioblastoma. In addition, various studies have found that diseases such as brain tumors, neurodegenerative diseases, cancer, and cardiovascular diseases are closely related to homocysteine. For these reasons, the importance of homocysteine and the need for diagnosis of diseases related to homocysteine are emerging. Methods for detecting homocysteine include high-performance liquid chromatography (HPLC), radiometric analysis, and photoluminescence (PL) immunoassay. However, because the measuring equipment is complex to use and bulky, it is difficult to use the same as equipment for on-site diagnosis of homocysteine.

The field of fluorescent molecular probes is a molecular unit tool used to analyze the properties or structures of molecules within a living body, and is convenient because it allows for the visual observation of fluorescence signals for investigating biological phenomena or detecting disease factors through various molecules and proteins present within a living body and their properties. Because of these characteristics, much development has been made in this field and it is widely utilized in various studies. In particular, this field may detect metabolites in a living body with high sensitivity and selectivity, and thus may be easily utilized for metabolites and protein molecules in real time: therefore, its importance is increasing.

To date, only a few fluorescent molecular probes capable of detecting biothiols, including homocysteine, have been reported. Although a variety of fluorescent molecular probes sensitive to cysteine and homocysteine have been reported, most of them have complex synthetic methods and lack selectivity for a specific type of biothiol. Due to the structural similarity between cysteine and homocysteine (the number of carbon atoms in cysteine=the number of carbon atoms in homocysteine+1), it is difficult to achieve selectivity, sensitivity, and specificity only for homocysteine. Furthermore, there is still a high level of difficulty in developing a fluorescent molecular probe that may selectively detect only homocysteine. Electrochemiluminescence probes with selectivity for homocysteine have also been developed: however, the synthesis method of the electrochemiluminescence probe is relatively complex and time-consuming and is unable to detect homocysteine at very low concentrations (for example, 0.176 ppm: 0.01 μM). In addition, it took more than 60 minutes to sense homocysteine and reach maximum phosphorescence intensity, making it impossible to detect homocysteine in a short period of time. The present inventors developed a fluorescent molecular probe compound capable of selectively detecting homocysteine with high sensitivity within a short period of time using a simple synthetic method, and completed the present disclosure by identifying the possibility of selectively detecting homocysteine present in plasma in vitro using the fluorescent molecular probe compound for early diagnosis of glioblastoma.

### DISCLOSURE

### Technical Problem

An aspect of the present disclosure is directed to providing a fluorescent probe compound capable of selectively detecting homocysteine in a short period of time and a method for measuring the homocysteine in plasma in vitro using the same.

Another aspect of the present disclosure is directed to providing a method for early diagnosis of glioblastoma using the fluorescent probe compound.

### Technical Solution

An embodiment of the present disclosure provides a compound represented by Formula 1 below:

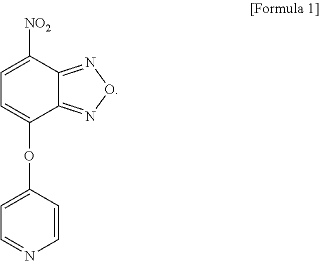 [SMILES: O=[N+]([O-])c1ccc(Oc2ccncc2)c2nonc12]

In an embodiment, the compound may react with homocysteine to exhibit a fluorescence turn-on phenomenon.

In an embodiment, the compound may exhibit the fluorescence turn-on phenomenon under conditions of pH 6 to 8.

In an embodiment, the compound may selectively emit fluorescence to homocysteine in a sample containing biothiols, metal ions or biomolecules. In an embodiment, the compound may be a fluorescent probe compound for selective detection of the homocysteine.

In addition, an embodiment of the present disclosure provides a chemical sensor for detecting homocysteine and a composition for detecting homocysteine, in which the composition includes the compound represented by Formula 1, a hydrate thereof, a solvate thereof, or a pharmaceutically acceptable salt thereof.

Furthermore, an embodiment of the present disclosure provides a method for detecting homocysteine, in which the method includes: adding the compound represented by Formula 1 to a sample (stage 1); irradiating the sample prepared in stage 1 with an excitation light source (stage 2); and measuring a change in emitted fluorescence (stage 3).

In addition, an embodiment of the present disclosure provides a composition for diagnosing a homocysteine-related disease, in which the composition includes the compound represented by Formula 1, a hydrate thereof, a solvate thereof, or a pharmaceutically acceptable salt thereof.

Furthermore, an embodiment of the present disclosure provides a method for providing information for diagnosing a homocysteine-related disease, in which the method includes: adding the compound represented by Formula 1 to a sample isolated from a human or an animal (stage 1); irradiating the sample prepared in stage 1 with excitation light (stage 2); and measuring a change in emitted fluorescence (stage 3). In addition, an embodiment of the present disclosure provides a method for preparing a compound represented by Formula 1 below, in which the method includes reacting triethylamine, 4-hydroxypyridine, and 4-chloro-7-nitrobenzofurazan:

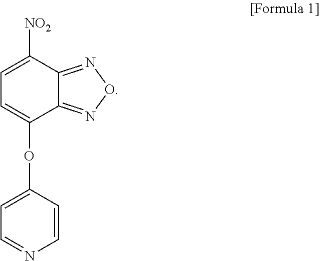 [SMILES: O=[N+]([O-])c1ccc(Oc2ccncc2)c2nonc12]

### Advantageous Effects

A fluorescent probe compound NPO-pyr of an embodiment of the present disclosure has a simple structure and can be synthesized in large quantities in a short period of time. The fluorescent probe compound can sense homocysteine in extracorporeal plasma isolated from blood, and can also detect the homocysteine in other bio-derived materials such as feces. In particular, the fluorescent probe compound can selectively detect the homocysteine by exhibiting fluorescence turning-on through a reaction in which coordination occurs specifically only to the homocysteine without interference from cysteine having a structure similar to the homocysteine. Accordingly, the complex structure and selectivity of fluorescent molecular probes for detecting cysteine and homocysteine have been overcome. With its rapid response speed, sensitivity, and selectivity, the fluorescent probe compound can be effectively utilized as an early diagnostic tool for homocysteine-related diseases, including glioblastoma.

### DESCRIPTION OF DRAWINGS

FIG. 1 is a graph showing the results of simultaneously identifying the changes in absorbance (Abs) and fluorescence (Emi) when Compound 1 (NPO-Pyr) according to an embodiment of the present disclosure reacts with homocysteine under the condition in aqueous solution (phosphate-buffered saline; PBS buffer, pH 7.4).

FIG. 2 is a graph showing the results of identifying the fluorescence change upon excitation at 460 nm after reaction with homocysteine (Hcy), cysteine (Cys), glutathione (GSH), and hydrogen sulfide (H₂S) under the condition in aqueous solution (pH 7.4, PBS buffer) for Compound 1 (NPO-Pyr) according to an embodiment of the present disclosure.

FIG. 3 is a graph showing the results of identifying the fluorescence change upon excitation at 460 nm after reaction of Compound 1 (NPO-Pyr) according to an embodiment of the present disclosure with homocysteine (Hcy), thiols, various metal ions, and biomolecules under the condition in aqueous solution (pH 7.4, PBS buffer).

FIG. 4 is a graph showing the results of identifying the fluorescence change upon excitation at 460 nm after reaction with homocysteine in aqueous solutions (pH 3 to 9) of different acidity for Compound 1 (NPO-Pyr) according to an embodiment of the present disclosure.

FIG. 5 shows the results of identifying the absorbance and fluorescence changes and the limit of detection (LOD) when Compound 1 (NPO-Pyr) according to an embodiment of the present disclosure reacts with homocysteine in the plasma (a, b, c) and feces (d, e, f) of the control group (normal mice) and the experimental group (glioblastoma mice).

FIG. 6 shows (a) brain imaging results of the experimental group (GBM) and the normal group (Normal) according to the time elapsed after the induction of glioblastoma (7 days, 14 days after the administration of glioblastoma cells) and (b) a fluorescence graph when Compound 1 (NPO-Pyr) according to an embodiment of the present disclosure reacts with homocysteine in the plasma of the control group and the experimental group (7 days, 14 days after the induction of glioblastoma) upon excitation at 460 nm.

FIG. 7 is a schematic representation showing that Compound 1 (NPO-Pyr) according to an embodiment of the present disclosure may selectively exhibit fluorescence turn-on properties by inducing coordination with homocysteine.

### MODES OF THE INVENTION

Hereinafter, an embodiment of the present disclosure will be described.

In an aspect, an embodiment of the present disclosure relates to a compound represented by Formula 1 below:

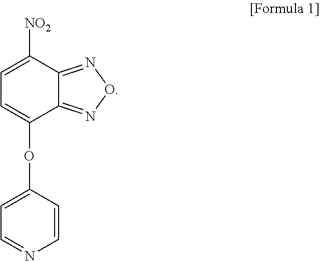 [SMILES: O=[N+]([O-])c1ccc(Oc2ccncc2)c2nonc12]

In an embodiment of the present disclosure, a compound represented by Formula 1 above, 4-nitro-7-(pyridine-4-yloxy)benzo[c] [1,2,5] oxadiazole, was synthesized and identified, and the compound was named NPO-pyr (see Example 1).

The compound represented by Formula 1 above of an embodiment of the present disclosure may also be used in the form of a hydrate thereof, a solvate thereof, or a pharmaceutically acceptable salt thereof.

The compound of an embodiment of the present disclosure may exhibit a fluorescence turn-on phenomenon by reacting with homocysteine, may react with homocysteine to emit fluorescence, and may be used for detecting homocysteine. Accordingly, the compound represented by Formula 1 above may be used as a fluorescent probe for detecting homocysteine.

The term “fluorescence turn-on” characteristic of an embodiment of the present disclosure means a characteristic in which no fluorescence is emitted in the absence of a detection target substance, or a weak fluorescence that may not be identified with the naked eye is emitted, but when the detection target substance is present, fluorescence is emitted by reacting with the detection target substance. The “fluorescence turn-on” characteristic is distinguished from the term “fluorescence-ratio” characteristic in that no fluorescence is emitted or a weak fluorescence that may not be identified with the naked eye is emitted before reacting with the detection target substance. The “fluorescence-ratio” characteristic means that fluorescence of a specific wavelength range is emitted even before reacting with the detection target substance, and the color of the fluorescence changes depending on the detection target substance.

For example, the “fluorescence turn-on” characteristic may be a fluorescence intensity that is at least 10 times or higher or 15 times or higher when reacting with a detection target substance than the fluorescence intensity emitted when the detection target substance is not present.

The “fluorescence turn-on” reference system of an embodiment of the present disclosure may complement the shortcomings of the “fluorescence-ratio” reference system. Specifically, the “fluorescence-ratio” reference system has the limitation that it is necessary to collect and analyze data by setting a reference wavelength. Furthermore, since fluorescence changes continuously, it is difficult to visually track the fluorescence change. In addition, when the spectrum before the fluorescence change and the spectrum after the fluorescence change overlap, the range of the fluorescence change is not large, and thus it is difficult to visually distinguish the fluorescence change. In contrast, in the case of the “fluorescence turn-on” reference system such as an NPO-pyr compound according to an example of the present disclosure, there is no fluorescence or only weak fluorescence that may not be identified with the naked eye, but clear fluorescence (at least 10-fold or higher increase in fluorescence intensity) occurs due to a detection target substance. Accordingly, it is possible to clearly visually determine whether the detection target substance is detected.

The fluorescence turn-on exhibited by the compound of an embodiment of the present disclosure may mean that fluorescence is not emitted before contact with homocysteine, but fluorescence is emitted upon contact and reaction with homocysteine.

The compound of an embodiment of the present disclosure may exhibit a fluorescence turn-on phenomenon through a reaction in which the thiol of homocysteine coordinates with the amine group in a pyridine molecule, and the amine group (—NH₂) of homocysteine binds by nucleophilic attack on C₁ of the pyridine portion of the compound. The mechanism by which the compound of an embodiment of the present disclosure acts as a fluorescent probe may be explained by FIG. 7 (see Example 8 and FIG. 7).

The compound of an embodiment of the present disclosure may exhibit a fluorescence turn-on phenomenon under conditions of pH 6 to 8, preferably may be capable of detecting homocysteine through fluorescence turning-on under conditions of pH 6.5 to 8, more preferably under conditions of pH 6.8 to 7.6, and may be most preferably under conditions of pH 7 to 7.5.

The compound of an embodiment of the present disclosure may be a fluorescent probe compound that selectively senses homocysteine by emitting selective fluorescence toward homocysteine in a sample containing biothiols, metal ions or biomolecules.

The sample may be water, a buffer solution, or a biological sample, and the biological sample may include cells, tissues, body fluids (saliva, etc.), blood, spinal fluid, cerebrospinal fluid, serum, plasma, urine, or feces isolated from humans or animals (rodents, mammals, etc.), and preferably plasma or feces, but is not limited thereto, and any biological sample that may be used in the relevant technical field may be used.

In an embodiment of the present disclosure, a new fluorescent molecular probe compound capable of selectively detecting homocysteine in a short period of time was developed, and the newly synthesized molecule was named NPO-Pyr (see Example 1). The NPO-pyr of an embodiment of the present disclosure showed stability in an aqueous solution (pH 7.4, PBS buffer), which is a biological composition, and was able to selectively react with homocysteine among amino thiols and biomolecules including cysteine having a structure similar to homocysteine in an aqueous solution (see Examples 2 to 5). The compound of an embodiment of the present disclosure exhibited a characteristic that allows coordination with homocysteine and reacts, and exhibited detection characteristics such as high sensitivity and specificity for homocysteine and a fast reaction time (within 5 minutes). In addition, it was identified that the compound selectively sensed homocysteine present in plasma isolated from mouse blood and exhibited fluorescence turning-on (see Examples 3, 4, and 6). Thus, it was identified that selective detection of homocysteine was possible using the compound NPO-pyr of an embodiment of the present disclosure. In addition, the compound reacted with homocysteine in the plasma of the control group (normal mice) and the experimental group (glioblastoma mice) and showed stronger fluorescence intensity in the experimental group than in the control group (see Example 7). Thus, an embodiment of the present disclosure was completed by identifying that glioblastoma was able to be distinguished using the compound NPO-pyr of an embodiment of the present disclosure.

In an aspect, an embodiment of the present disclosure provides a chemical sensor for detecting homocysteine, in which the chemical sensor includes a compound represented by Formula 1 above, a hydrate thereof, a solvate thereof, or a pharmaceutically acceptable salt thereof.

In addition, an embodiment of the present disclosure provides a composition for detecting homocysteine, in which the composition includes a compound represented by Formula 1 above, a hydrate thereof, a solvate thereof, or a pharmaceutically acceptable salt thereof.

The chemical sensor or composition for detecting homocysteine of an embodiment of the present disclosure may selectively detect homocysteine in a biological sample.

The composition for detecting homocysteine may further include at least one selected from a solvent, an acid, a base, and a buffer solution. The composition for detecting homocysteine may be prepared by adding the aforementioned compound to a solvent, a buffer solution or a mixture thereof, and adding an acid and/or a base thereto. In addition, the composition for detecting homocysteine may additionally include other additives that may be used in the pertinent technical field. The contents of the solvent, acid, base, and buffer solution contained in the composition may be appropriately adjusted depending on the required performance. The solvent may include water, THF, methanol, ethanol, HI aqueous solution, N,N-dimethylformamide, or a combination thereof. Alternatively, the composition may be mixed with a biological sample. The biological sample may include cells, tissues, body fluids (saliva, etc.), blood, spinal fluid, cerebrospinal fluid, serum, plasma, urine or feces isolated from humans or animals (rodents, mammals, etc.), and preferably plasma or feces, but is not limited thereto, and any biological sample that may be used in the pertinent technical field may be used.

In an aspect, an embodiment of the present disclosure provides a method for detecting homocysteine, in which the method includes: adding a compound represented by Formula 1 above to a sample (stage 1); irradiating an excitation light source to the sample prepared in stage 1 (stage 2); and measuring a change in emitted fluorescence (stage 3).

In the method for detecting homocysteine according to an embodiment of the present disclosure, the sample is as described above.

In the method for detecting homocysteine according to an embodiment of the present disclosure, the excitation light source may be a light source having an excitation wavelength of 440 to 480 nm, and preferably, an excitation wavelength of 450 to 470 nm. Herein, it is preferable to irradiate the light source using a fluorescence spectrophotometer or UV absorption spectroscopy.

In the method for detecting homocysteine according to an embodiment of the present disclosure, stage 3 may include identifying that changes in absorption and fluorescence emission spectra occur as a result of the compound represented by Formula 1 above according to an embodiment of the present disclosure selectively reacting with homocysteine. Preferably, the fluorescence emission and fluorescence intensity may increase in the presence of homocysteine.

In an aspect, an embodiment of the present disclosure provides a composition for diagnosing a homocysteine-related disease, in which the composition includes a compound represented by Formula 1 above, a hydrate thereof, a solvate thereof, or a pharmaceutically acceptable salt thereof.

In the diagnostic composition according to an embodiment of the present disclosure, the homocysteine-related disease may be selected from the group consisting of brain tumor, neurodegenerative disease, cancer, and cardiovascular disease, and preferably may be glioblastoma.

The diagnostic composition according to an embodiment of the present disclosure may diagnose glioblastoma at an early stage by detecting homocysteine present in a biological sample, preferably plasma or feces, more preferably in vitro plasma.

In an aspect, an embodiment of the present disclosure relates to a method for providing information for diagnosing a homocysteine-related disease, in which the method includes: adding the compound represented by Formula 1 above to a sample isolated from a human or an animal (stage 1); irradiating excitation light to the sample prepared in stage 1 (stage 2); and measuring a change in emitted fluorescence (stage 3).

The homocysteine-related disease may be selected from the group consisting of brain tumor, neurodegenerative disease, cancer, and cardiovascular disease, and preferably may be glioblastoma.

In the method for providing information for diagnosis according to an embodiment of the present disclosure, the sample may include cells, tissues, body fluids (saliva, etc.), blood, spinal fluid, cerebrospinal fluid, serum, plasma, urine or feces, etc. isolated from a human or an animal (rodent, mammal, etc.), preferably plasma or feces, but is not limited thereto, and any biological sample that may be used in the pertinent technical field may be used.

In the method for providing information for diagnosis according to an embodiment of the present disclosure, the excitation light source may be a light source having an excitation wavelength of 440 to 480 nm, and preferably, an excitation wavelength of 450 to 470 nm. Herein, it is preferable to irradiate the light source using a fluorescence spectrophotometer or UV absorption spectroscopy, but is not limited thereto.

In the method for providing information for diagnosis according to an embodiment of the present disclosure, stage 3 may include identifying that the fluorescence intensity of the sample to be measured has increased compared to the fluorescence intensity of the sample before adding the compound or the normal control sample.

More specifically, stage 3 may include identifying that changes in absorption and fluorescence emission spectra occur as the compound represented by Formula 1 above according to an embodiment of the present disclosure selectively reacts with homocysteine. Preferably, the fluorescence turning-on and fluorescence intensity may increase in the presence of homocysteine, and when an increase in fluorescence emission and fluorescence intensity is identified in the sample to be measured compared to the sample before adding the compound according to an embodiment of the present disclosure or the normal control sample, it may be determined that glioblastoma is induced.

In an aspect, an embodiment of the present disclosure provides a method for preparing a compound represented by Formula 1 below, in which the method includes reacting triethylamine, 4-hydroxypyridine and 4-chloro-7-nitrobenzofurazan:

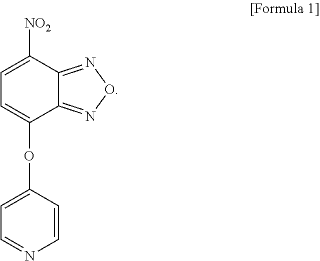 [SMILES: O=[N+]([O-])c1ccc(Oc2ccncc2)c2nonc12]

In the preparation method according to an embodiment of the present disclosure, the reaction may be performed using N,N-dimethylformamide (DMF) from which oxygen has been removed with nitrogen as a reaction solvent.

Preferably, triethylamine and 4-hydroxypyridine may be added to DMF to cause a first reaction, and then DMF may be added again and 4-chloro-7-nitrobenzofuran may be added to cause a second reaction.

In the preparation method according to an embodiment of the present disclosure, after the above stage, there may be further included mixing ethyl acetate (EtOAc) and water into the reacted solution, and then separately recovering only an organic layer (EtOAc layer) and drying and concentrating the same. In addition, a process of purifying the same using column chromatography or the like may be additionally included.

In the preparation method according to an embodiment of the present disclosure, the preparation process of the compound represented by Formula 1 above may be represented as in Reaction Formula 1 of Example 1 below.

Hereinafter, an embodiment of the present disclosure will be described in more detail by the following examples. However, these examples are only intended to illustrate an embodiment of the present disclosure, and the scope of the present disclosure is not limited by these examples.

### EXAMPLES

### Example 1. Synthesis and Structural Analysis of Compounds for Detecting Homocysteine in Plasma

The present inventors designed 4-chloro-7-nitrobenzofurazan with 4-hydroxypyridine, an aromatic ring compound, as a functional group in order to rapidly measure homocysteine in plasma, which is one of bio-derived materials. In detail, the molecular structure was designed so that the thiol group of homocysteine coordinates with the amine group of pyridine, and then the amine group of homocysteine attacks C₁ of Compound 1 as a nucleophile, and Compound 1 (4-nitro-7-(pyridine-4-yloxy)benzo[c] [1,2,5] oxadiazole) was synthesized according to Reaction Formula 1 below.

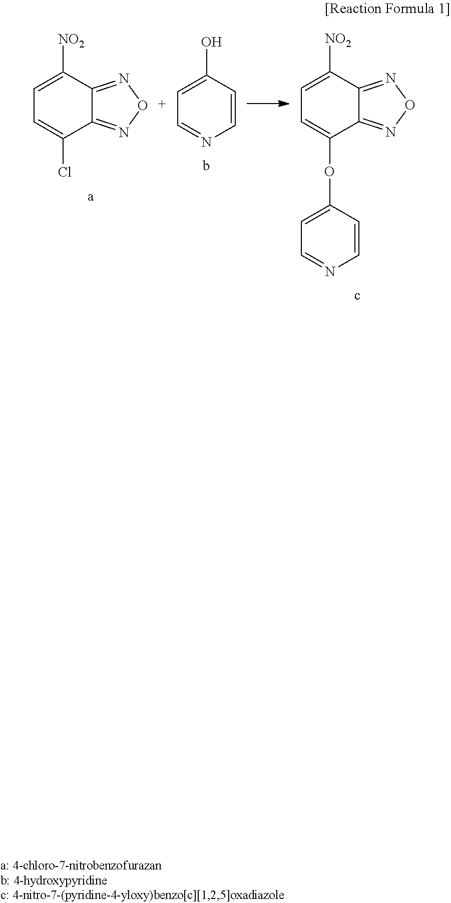

### 1-1. Synthesis of 4-nitro-7-(pyridine-4-yloxy)benzo[c] [1.2.5] oxadiazole

Specifically, in Reaction Formula 1 above, triethylamine (36.6 μL, 0.50257 mmol, TCI, S0377) and 4-hydroxypyridine (44.7 mg, 0.50257 mmol, TCI, 626-64-2) were dissolved in 3 mL of N,N-dimethylformamide (DMF) from which oxygen had been removed with nitrogen, and the mixture was stirred at 0° C. for 30 minutes (revolution per minute: 250 rpm) using a stirring bar so that the compounds might be well mixed. Thereafter, the synthetic starting material, 4-chloro-7-nitrobenzofurazan (50 mg, 0.00025 mol, TCI, A55921G) and 3 mL of N,N-dimethylformamide (DMF) from which oxygen had been removed with nitrogen were added, and the temperature was raised to 25° C. in a silicon oil container. When the temperature reached 25° C., the mixture was stirred for 30 minutes and then the reaction was stopped with water (6 mL). To extract the organic layer, a separatory funnel was used to mix ethyl acetate (EtOAc) and water in a 1:1 ratio, and through this process, only the organic layer was able to be obtained separately. Thereafter, the organic layer was dried with anhydrous sodium sulfate (Na₂SO₄, 2 g) and concentrated using a rotary evaporator (37° C., 20 to 500 mmHg). The dark brown solid compound thus obtained was separated using column chromatography (6 cm in diameter, 15 cm in height) using silica gel (Merck-silica gel 60, 230 to 400 mesh) (Elution solution: MeOH/DCM=5/95) to obtain a yellow solid compound (Compound 1, 28.1 mg, 63.2%).

Nuclear magnetic resonance (NMR) analysis was performed to identify that the synthesis was successful. The analysis identified that Compound 1 was successfully synthesized. The NMR analysis results are as follows:
1H-NMR (500 MHZ, Acetonitrile-D₃) δ 8.67 (d, J=8.0 Hz, 1H), 8.11 (dd, J=6.0, 2.0 Hz, 2H), 7.66 (d, J=8.0 Hz, 1H), 6.41 (dd, J=6.0, 2.0 Hz, 2H). 13C-NMR (126 MHZ, Acetonitrile-D₃) δ 148.1, 145.8, 140.6, 138.3, 134.1, 122.9, 120.4, 2.3, 2.1, 1.9, 1.8, 1.6.

In this connection, Compound 1 was identified as ‘4-nitro-7-(pyridine-4-yloxy)benzo[c] [1,2,5] oxadiazole’ and named ‘NPO-pyr’.

### Example 2. Identification of Fluorescence Emission Characteristics of Compound 1 (NPO-pyr)

In order to identify the fluorescence emission characteristics of Compound 1 (NPO-pyr) prepared in Example 1 above, the absorbance and fluorescence emission graphs were measured after reaction with homocysteine under an aqueous solution of neutral conditions (pH 7.4, PBS buffer).

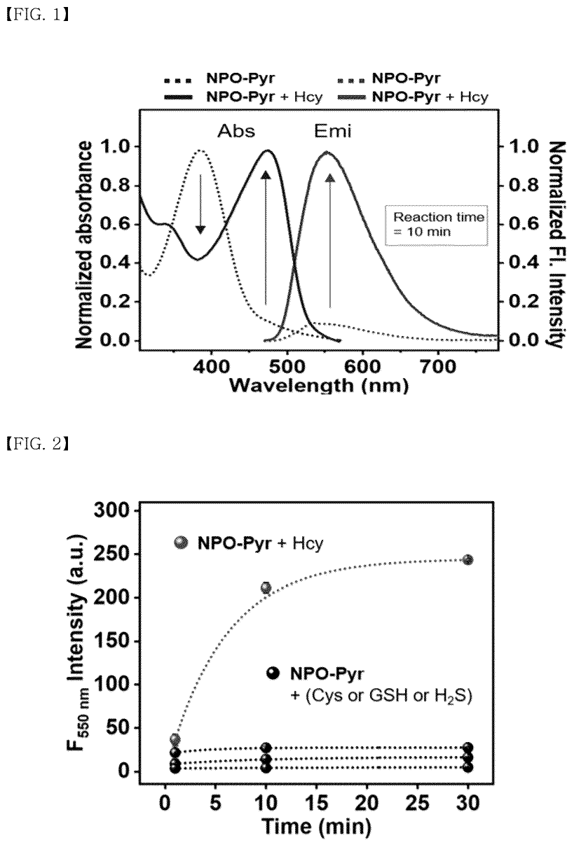

Specifically, Compound 1 (10 μM) and homocysteine (DL-homocysteine, 10 μM) were stirred in an aqueous solution (pH 7.4, PBS buffer) for 10 minutes, and then the fluorescence emission and absorbance values were measured (excitation wavelength: 460 nm). For analysis of the absorption spectrum (UV/Vis absorption spectra), a UV/Vis spectrophotometer (Agilent Technologies Cary 8454, USA) was used, and for analysis of the fluorescence spectra, a fluorophotometer (SHIMADZU CORP. RF-6000, Japan) was used. In this connection, the cell that puts the compound into each device was a standard quartz cell with a thickness of 1 cm (internal volume=0.1 cm). The measured results are shown in FIG. 1.

FIG. 1 shows the results of changes in absorbance (Abs) and fluorescence emission (Emi) graphs according to the reaction between Compound 1 and homocysteine under an aqueous solution of neutral conditions (pH 7.4, PBS buffer). It was identified that the absorbance spectrum increased at 488 nm and the fluorescence emission spectrum increased at 550 nm. In addition, it was identified that the fluorescence turn-on phenomenon occurred around 550 nm as Compound 1 reacted with homocysteine.

### Example 3. Identification of Homocysteine Reaction Time and Selectivity of Compound 1 (NPO-pyr)

In order to identify whether Compound 1 (NPO-pyr) would react with various types of aminothiols including homocysteine under neutral conditions (pH 7.4), Compound 1 and aminothiol were added to an aqueous solution of neutral conditions (pH 7.4, PBS buffer) and the fluorescence emission graph was measured.

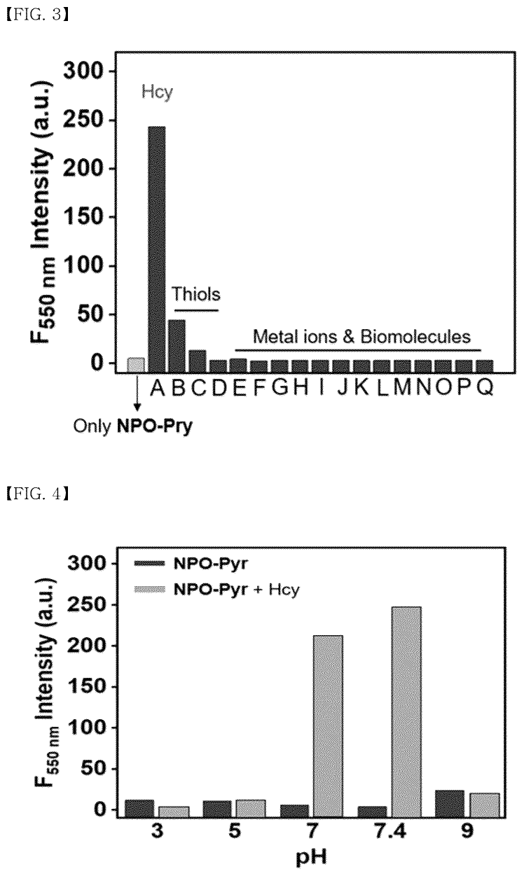

Specifically, Compound 1 (10 μM) was reacted with homocysteine (DL-homocysteine, 10 μM), cysteine (10 μM), glutathione (L-glutathione, 10 μM), and hydrogen sulfide (10 μM) in an aqueous solution (pH 7.4, PBS buffer), and the fluorescence value was measured (excitation wavelength: 460 nm). Amino thiols including homocysteine were purchased from Sigma, TCI, and a fluorophotometer (SHIMADZU CORP. RF-6000, Japan) was used for fluorescence spectra analysis. In this connection, the cell that puts the compound into each device was a standard quartz cell with a thickness of 1 cm (internal volume=0.1 cm). The measured results are shown in FIG. 2.

FIG. 2 is a graph showing the reaction of Compound 1 with amino thiol over time (0 to 30 minutes) under aqueous solution conditions (pH 7.4, PBS buffer). An increase in fluorescence of Compound 1 was observed within 5 minutes after the addition of homocysteine, and the fluorescence intensity was maintained for up to 30 minutes, whereas no fluorescence emission was observed for other amino thiols except homocysteine, identifying its low reactivity. Thus, it was identified that Compound 1 had the highest selectivity and fastest reaction time for homocysteine among various amino thiols under aqueous solution conditions (pH 7.4, PBS buffer).

### Example 4. Identification of Selectivity of Compound 1 (NPO-pyr) toward Biomolecules

It was identified through fluorescence changes that Compound 1 (NPO-pyr) exhibited selectivity only for homocysteine among various types of biomolecules.

Specifically, in order to identify whether Compound 1 was selective for homocysteine, fluorescence-turning on was determined. An aqueous solution (pH 7.4, PBS buffer) was used, and metal ions were purchased from Aldrich, Oriental Chemicals, Samchun chemicals, and Duksan chemicals. Compound 1 was dissolved in dimethyl sulfoxide (DMSO) at 10 mM and used, and the amount of dimethyl sulfoxide was controlled to be the same in each solvent (less than 1%) under the final solvent conditions used. The concentration of Compound 1 was 10 μM, and the biomolecule was treated identically at 10 μM. The excitation wavelength was 460 nm, and the fluorescence value at a fluorescence wavelength of 550 nm was measured using a fluorescence spectrophotometer, and is shown in FIG. 3.

The graph in FIG. 3 shows that the fluorescence intensity was measured after treating various biomolecules and ions (anion) in an aqueous solution (pH 7.4, PBS buffer) containing Compound 1. In the graph of FIG. 3, the result of treating only Compound 1 is represented by a light gray bar graph, and the result of reacting Compound 1 with a biomolecule including homocysteine (hcy) is represented by a dark gray bar graph. The types of ions indicated on the horizontal axis in the graph of FIG. 3 are as follows:
(A) Probe 1 with DL-Homocysteine;
(B) Probe 1 with L-Cysteine;
(C) Probe 1 with L-Glutathion;
(D) Probe 1 with Hydrogen sulfide;
(E) Probe 1 with Bovine Serum Albumin;
(F) Probe 1 with Holmium (III) chloride;
(G) Probe 1 with Iron (II) chloride;
(H) Probe 1 with Iron (III) chloride;
(I) Probe 1 with Magnesium chloride;
(J) Probe 1 with Nikel (II) chloride;
(K) Probe 1 with Phosphorus pentachloride;
(L) Probe 1 with Gold trichloride;
(M) Probe 1 with Palladium (II) chloride;
(N) Probe 1 with potassium chloride;
(O) Probe 1 with Silver nitrate;
(P) Probe 1 with Sodium chloride; and
(Q) Probe 1 with Cobalt chloride.

As a result, as shown in the graph of FIG. 3, NPO-pyr (Only NPO-pyr) showed almost no fluorescence intensity value, but A, where homocysteine is present, showed a strong fluorescence intensity value. In contrast, NPO-pyr showed little fluorescence turn-on phenomenon under amino thiol, metal ion, and biomolecule conditions other than homocysteine. Thus, it was identified that Compound 1 had high selectivity for homocysteine.

### Example 5. Identification of pH-Dependent Homocysteine Sensing Ability of Compound 1 (NPO-pyr)

The present inventors identified the homocysteine sensing ability of Compound 1 (NPO-pyr) according to acidity (pH).

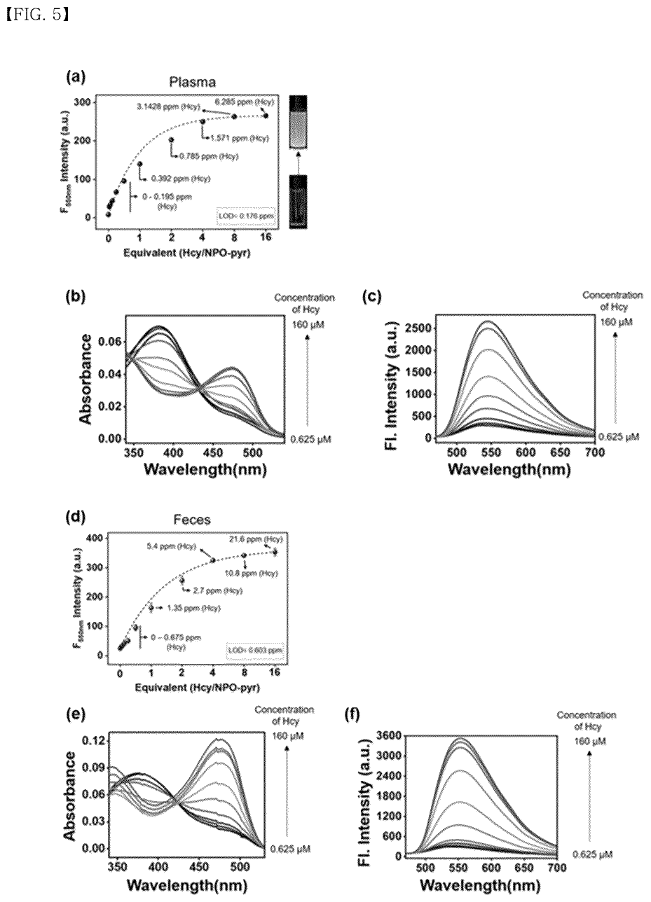

Specifically, in order to observe the sensitivity of acidity (pH) to homocysteine based on the fluorescence turning-on of Compound 1, the sensitivity was identified under various acidity conditions (pH 3 to 9) while fixing the concentrations of Compound 1 (10 μM) and homocysteine (10 μM). In other words, 10 UM of homocysteine was treated under the conditions of pH 3, 5, 7, 7.4, and 9, respectively, and 10 μM of Compound 1 was stirred for 10 minutes, and then the fluorescence intensity was measured. In this connection, an excitation wavelength of 460 nm was used, and a fluorescence emission wavelength of 550 nm was measured. The fluorescence intensity is shown in the bar graph in FIG. 4.

As a result, as shown in FIG. 4, it was identified that the reaction with homocysteine was most sensitive at pH 7 and 7.4, and relatively weak at other pHs, thereby identifying that NPO-pyr was able to be applied to detecting homocysteine in biological samples.

### Example 6. Identification of Homocysteine Sensing Ability of Compound 1 (NPO-pyr) in Mouse Plasma and Feces

In order to identify the detection ability of Compound 1 (NPO-pyr) for homocysteine present in mouse plasma and feces, plasma (20 μL) isolated from blood and an extract (9.5 mg) extracted from mouse feces were dissolved in an aqueous solution (DI. H₂O) (5 mL), treated with homocysteine at various concentrations (1 to 160 ng/μL), and then treated with Compound 1 (10 μM). After stirring for 10 minutes in a stirrer at 37° C. for reaction, the fluorescence spectrum was measured (excitation wavelength: 460 nm). Compound 1 was dissolved in DMSO solution at μM and used, and the amount of DMSO was ensured to be the same in each container (less than 1%) under the final solvent conditions used.

As a result, as shown in FIGS. 5A to 5F, the ability to sense homocysteine in mouse plasma was identified to be higher, and the LOD value was calculated as 0.176 ppm (=0.01 μM). In addition, the potential to sense homocysteine not only in mouse plasma but also in mouse feces was identified.

### Example 7. Identification of Differences in Fluorescence Intensity of Compound 1 (NPO-pyr) in Plasma of Glioblastoma Mice

In order to verify the homocysteine sensing ability of Compound 1 (NPO-pyr) according to an embodiment of the present disclosure, normal mice were used as a control group and glioblastoma mice were used as an experimental group, and the difference in fluorescence intensity of Compound 1 in the plasma of each thereof and the degree of glioblastoma progression were analyzed.

First, in order to prepare the experimental group, a glioblastoma mouse model was created by administering a solution containing 5.0×10⁵ cells in 5 μL of cell culture media without fetal bovine serum (FBS) using LUC-U87MG cells (glioblastoma cells) to the mouse brain (thalamus).

Thereafter, in order to determine the degree of glioblastoma progression through mouse brain imaging, luciferin (D-Luciferin, 150 mg/kg, n=10) was administered intraperitoneally to mice, and the fluorescent emission signal (intensity) of glioblastoma was identified using a fluorescence tissue imaging system (FTIS: VISQUE In Vivo Elite. Vieworks).

In order to identify the reaction between Compound 1 and mouse plasma, a fluorescence measuring device (Spectro-fluorophotometer: SHIMADZU CORP. RF-6000, Japan) was used. The glass cuvette used for fluorescence measurements was a standard quartz cell with a thickness of 1 cm on all sides (internal volume=0.1 cm, Hellma Analytics, Jena, Germany).

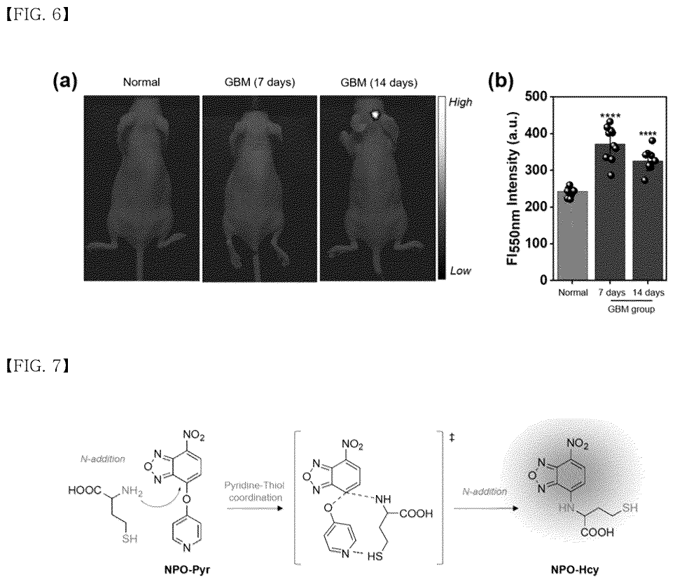

In addition, in order to identify the reaction between Compound 1 and mouse plasma, blood was collected from the control group and the experimental group, the plasma was isolated, reacted with Compound 1, and then the fluorescence intensity was measured at an emission wavelength of 550 nm using a fluorescence measuring device (Spectro-fluorophotometer; SHIMADZU CORP. RF-6000, Japan) (excitation wavelength: 460 nm). The glass cuvette used for fluorescence measurements was a standard quartz cell with a thickness of 1 cm on all sides (internal volume=0.1 cm, Hellma Analytics, Jena, Germany). The results of mouse brain imaging are shown in FIG. 6A, and the difference in fluorescence intensity of Compound 1 in the plasma of the control group and the experimental group is shown as a bar graph in FIG. 6B.

As a result, as shown in FIG. 6A, it was identified that the fluorescence emission intensity increased over time (7 and 14 days after administration of glioblastoma cells) in glioblastoma mice, indicating that glioblastoma was progressing. In addition, as shown in FIG. 6B, it was identified that the plasma of the experimental group reacted with Compound 1 compared to the control group showed a stronger fluorescence intensity. As a result, the ability of NPO-pyr to sense homocysteine in plasma was identified. In addition, it was identified that NPO-pyr was able to be utilized for the early diagnosis of glioblastoma using the same.

### Example 8. Schematic Diagram of Reaction Mechanism of Homocysteine of Compound 1 (NPO-pyr)

FIG. 7 schematically illustrates the homocysteine sensing mechanism of Compound 1 (NPO-pyr) prepared in Example 1 of the present disclosure. The mechanism proposed in an embodiment of the present disclosure is that NPO-pyr and homocysteine are coordinated, and the amine group (—NH₂) of homocysteine is added to the pyridine portion of NPO-pyr, thus binding to NPO-pyr. In addition, the coordination between NPO-pyr and homocysteine may be induced to selectively exhibit fluorescence turn-on characteristics.

The above description of the present disclosure is provided for the purpose of illustration, and it would be understood by those skilled in the technical field to which the present disclosure pertains that various changes and modifications may be made without changing technical ideas or essential features of the present disclosure. Thus, it is clear that the above-described embodiments are illustrative in all aspects and do not limit the present disclosure.

1. A compound represented by Formula 1 below:

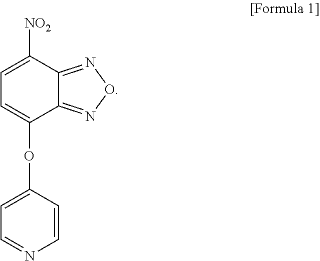 [SMILES: O=[N+]([O-])c1ccc(Oc2ccncc2)c2nonc12]

2. The compound of claim 1, wherein the compound reacts with homocysteine to exhibit a fluorescence turn-on phenomenon.

3. The compound of claim 2, wherein the compound exhibits the fluorescence turn-on phenomenon under conditions of pH 6 to 8.

4. The compound of claim 1, wherein the compound selectively emits fluorescence to homocysteine in a sample containing biothiols, metal ions or biomolecules.

5. The compound of claim 4, wherein the sample is water, a buffer solution, or a biological sample.

6. A chemical sensor for detecting homocysteine, the chemical sensor comprising the compound of claim 1, a hydrate thereof, a solvate thereof, or a pharmaceutically acceptable salt thereof.

7. The chemical sensor of claim 6, wherein the chemical sensor selectively detects the homocysteine in a biological sample.

8. A composition for detecting homocysteine, the composition comprising the compound of claim 1, a hydrate thereof, a solvate thereof, or a pharmaceutically acceptable salt thereof.

9. The composition of claim 6, wherein the composition selectively detects the homocysteine in a biological sample.

10. A method for detecting homocysteine, the method comprising:
adding the compound of claim 1 to a sample (stage 1);
irradiating the sample prepared in stage 1 with an excitation light source (stage 2); and
measuring a change in emitted fluorescence (stage 3).

11. A composition for diagnosing a homocysteine-related disease, the composition comprising the compound of claim 1, a hydrate thereof, a solvate thereof, or a pharmaceutically acceptable salt thereof.

12. The composition of claim 11, wherein the homocysteine-related disease is selected from the group consisting of brain tumor, neurodegenerative disease, cancer, and cardiovascular disease.

13. The composition of claim 11, wherein the homocysteine-related disease is glioblastoma.

14. A method for providing information for diagnosing a homocysteine-related disease, the method comprising:
adding the compound of claim 1 to a sample isolated from a human or an animal (stage 1);
irradiating the sample prepared in stage 1 with excitation light (stage 2); and
measuring a change in emitted fluorescence (stage 3).

15. A method for preparing a compound represented by Formula 1 below, the method comprising reacting triethylamine, 4-hydroxypyridine, and 4-chloro-7-nitrobenzofurazan: 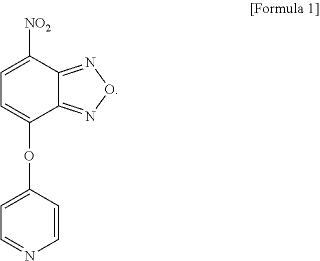 [SMILES: O=[N+]([O-])c1ccc(Oc2ccncc2)c2nonc12]
